(19) United States
(12) Patent Application Publication
(10) Pub. No.: US 2005/0180971 A1
(43) Pub. Date: Aug. 18, 2005

(54) Cancer therapy

(76) Inventor: Martin Leonard Ashdown, Thornbury (AU)

Correspondence Address: FAEGRE & BENSON LLP;PATENT DOCKETING, 2200 WELLS FARGO CENTER, MINNEAPOLIS, MN, 55402, US

(21) Appl. No.: 10/503,794

(22) PCT Filed: Feb. 14, 2003
(86) PCT No.: PCT/AU03/00187

(30) Foreign Application Priority Data
Feb. 14, 2002 (AU) PS 0547

(51) Int. Cl. A61K 39/395; A61K 35/78; A61K 31/4745

(52) U.S. Cl. 424/144.1; 424/725; 514/283; 600/1; 606/1

(57) ABSTRACT

The present invention provides methods for treating a cancer patient. The methods rely on the observation that cells of the immune system which target a tumor cell (“effector cells”) clonally expand before a population of “regulator” or “suppressor” cells which down-regulate the activity of the immune cells which target the tumor cell. Methods are provided herein which stimulate effector cell production and also provides means for inhibiting the production of, limiting the function of, and/or destroying, regulator cells, in the treatment of cancer.

### FIELD OF THE INVENTION

The present invention relates to methods for treating cancer. In particular, the present invention relates to an immunomodulation which results in the destruction of tumor cells.

### BACKGROUND OF THE INVENTION

Although our understanding of the mechanisms and possible treatment of cancer has increased over recent years, cancer remains a major cause of death throughout the developed world. Non-specific approaches to cancer management, such as surgery, radiotherapy and generalized chemotherapy, have been successful in the management of some circulating and slow-growing solid cancers. However, many types of cancer are generally highly resistant to standard treatments. Accordingly, there is a need for further, and more effective, cancer therapies.

Immunotherapy has also been investigated as an avenue for treating cancer. The general principle of immunotherapy is to confer upon the subject being treated an ability to mount what is in effect a rejection response, specifically against the neoplastic cells. There are a number of immunological strategies under development including, adoptive immunotherapy using stimulated autologous cells of various kinds; systemic transfer of allogeneic lymphocytes; intra-tumor implantation of immunologically reactive cells; and vaccination at a distant site to generate a systemic tumor-specific immune response.

Adoptive T-cell therapy involves the passive transfer of antigen-reactive T cells to a tumor-bearing host in order to start tumor rejection. The cells are histocompatible with the subject, and are generally obtained from a previous autologous donation.

One approach to adoptive immunotherapy is to stimulate autologous lymphocytes ex vivo with tumor-associated antigen to make them tumor-specific. Autologous lymphocytes and killer cells may also be stimulated non-specifically. In one example, Fc receptor expressing leukocytes that can mediate an antibody-dependent cell-mediated cytotoxicity reaction are generated by culturing with a combination of IL-2 and IFN-γ (U.S. Pat. No. 5,308,626). In another example, peripheral blood-derived lymphocytes cultured in IL-2 form lymphokine-activated killer (LAK) cells, which are cytolytic towards a wide range of neoplastic cells, but not normal cells. LAK are primarily derived from natural killer cells expressing the CD56 antigen, but not CD3. Such cells can be purified from peripheral blood leukocytes by IL-2-induced adherence to culture plates (A-LAK cells; see U.S. Pat. No. 5,057,423).

Adoptive transfer of allogeneic lymphocytes relies on the creation of a general level of immune stimulation, and thereby overcomes the anergy that prevents the host's immune system from rejecting the tumor. Even though initial experiments were conducted over a decade ago, the strategy has not gained general acceptance, especially for the treatment of solid tumors.

Intra-tumor implantation is a strategy directed to delivering immune cells against the tumor directly to the site of action. Since the transplanted cells do not circulate, they need not be histocompatible with the host. Intratumor implantation of allogeneic cells may promote the ability of the transplanted cells to react with the tumor, and initiate a potent graft versus tumor response. For example, Kruse et al. (1990) demonstrated that direct intratumoral implantation of allogeneic cytotoxic T lymphocytes (CTL) into brain tumors growing in Fischer rats resulted in a significant survival advantage over other populations of lymphocytes, including syngeneic CTL, LAK cells, adherent-LAK cells or IL-2 alone.

Finally, the generation of an active systemic tumor-specific immune response of host origin has also been used in attempts to control cancer growth. The response is elicited from the subject's own immune system by administering a vaccine composition at a site distant from the tumor. The specific antibodies or immune cells elicited in the host as a result will hopefully migrate to the tumor, and then eradicate the cancer cells, wherever they are in the body. Various types of vaccines have been proposed, including isolated tumor-antigen vaccines and anti-idiotype vaccines. An alternative approach to an anti-tumor vaccine is to use tumor cells from the subject to be treated, or a derivative of such cells. However, in many patients tumor regression has not been detected in response to their own tumor antigen, even when comprised in a vaccine preparation.

In some approaches to increase immunogenicity, autologous or syngeneic tumor cells are genetically altered to produce a costimulatory molecule. Examples of costimulatory molecules include cell surface receptors, such as the B7-1 costimulatory molecule, allogeneic histocompatibility antigens or cytokines.

Populations of CD4+ T cells have been shown to regulate self-reactive T cells, and hence guard against autoimmune diseases (Sakaguchi et al., 2001). The inability to remove or control such self-reactive cells plays a role in many autoimmune diseases. However, the ability of the mammalian immune system to typically control such self-reactive lymphocytes limits the capability of the immune system to control tumor growth as the tumor is expressing self antigens. Many tumor antigens can be considered as autoantigens as they are often derived from the proteins of fetal development. Thereby, the immune response in the cancer patient against the cancer should be considered an autoimmune response. This response is self limiting through regulatory mechanisms.

Despite advances in cancer therapy, particularly cancer immunotherapy, there remains a need for effective methods for treating cancer. The present invention provides an alternative immunotherapy that can be used to treat cancer

### SUMMARY OF THE INVENTION

The present inventor has noted that at least two populations of immune cells are produced in response to the presence of a tumor antigen. More particularly, the immune system of a mammal suffering from uncontrolled cell growth (cancer) is capable of mounting an immune response against the tumor through a group of cells herein generally referred to as “effector cells”, however, a second population of cells are also produced which regulate the “effector cells”, herein generally referred to as “regulator cells”, limiting the mammal's ability to effectively control or eradicate the tumor.

Further, it has also been noted that the relative number of effector cells expand in response to a tumor antigen before the regulator cells expand. This provides an opportunity to prevent the production of, limit the function of, or destroy, the “regulator cells” whilst maintaining the “effector cells”.

Although in some cases it is possible to screen for individuals that may be predisposed to cancer because of inherited genetic defects, the initial immune response to a tumor antigen produced by a neoplastic cell is difficult to trace as tumor growth can only be detected after the event has commenced. In fact, in many instances tumor growth is well advanced before detection.

Whilst effector cells are produced by the mammalian immune system which are capable of controlling tumor growth, their activity is compromised by the production of regulator cells which have a negative impact upon the effector cell population. In order to optimize effector cell function the present inventor has devised methods of cancer therapy that at least partially “reset” the immune response to a tumor antigen, thus providing a means to take advantage of the “effector cell” population to control tumor growth.

Accordingly, in a first aspect the present invention provides a method of treating cancer in a mammalian subject, the method comprising
i) reducing tumor load in the subject,
ii) allowing the number and/or activity of effector cells, directed against a tumor antigen, to increase in response to tumor antigen based stimulation of effector cells, and
iii) subsequently administering to the subject an agent which inhibits the production of, limits the function of, and/or destroys, regulator cells,
   wherein the timing of administration of the agent is selected such that the activity of the effector cells is not significantly reduced.

Upon the conclusion of step i), tumor cell proliferation will recommence if any tumor cells remain, which in turn will lead to an expansion of effector cells directed against a tumor antigen(s) produced by the tumor cells.

Following the reduction in the subjects tumor load, the subject will need to be monitored to determine when the agent should be administered. Factors which can be monitored include, but are not limited to, tumor antigen levels, CD8+CD4-T cell levels, CD4+CD8-T cell levels, and acute phase inflammatory markers such as c-reactive protein levels.

In a preferred embodiment, fluctuations in the levels of c-reactive protein in the subject are monitored to determine when the agent is administered. Preferably, the agent is administered approximately when the levels of c-reactive protein begin to decrease. In this instance, effector cell production and/or activity results in increased levels of c-reactive protein. Upon clonal expansion of regulator cells, the activity of effector cells is downregulated resulting in a decrease in c-reactive protein levels.

In a preferred embodiment of the first aspect, the agent is administered approximately when CD8+CD4-T cell numbers have peaked in response to tumor antigen based stimulation of effector cells following step i).

Since tumor antigen based stimulation following step i) results in the production of effector cells, these cells will stablize and/or result in a decrease in tumor cell numbers before regulator cells are produced. Accordingly, in a further preferred embodiment, the agent is administered approximately when the tumor cell numbers are decreasing and when regulator cell numbers are increasing. Typically, tumor cell numbers are determined by analysing the levels of an appropriate tumor cell marker.

Naturally, if surgery is conducted it will be desirable to attempt to remove all of the tumor cells. However, this is sometimes not possible due to the location of the tumor, and/or at least some tumor cells are missed. In a preferred embodiment, the tumor load is reduced in the subject by removing at least some of the cancer cells by surgery. Preferably, at least 50%, more preferably at least 75%, more preferably at least 90% and even more preferably at least 95% of the tumor cells are removed by surgery.

In an alternate preferred embodiment, the tumor load is reduced by administering an anti-cancer compound.

The anti-cancer compound can be any known molecule that either specifically or non-specifically (for example, general anti-mitotic drugs) destroys, or limit the function of, tumor cells in the subject. Preferred anti-cancer compounds include, but are not limited to, vinblastine and anhydro vinblastine.

In a further alternate preferred embodiment, the tumor load is reduced by exposing the subject to radiotherapy.

The production of effector cells could be enhanced by the administration of an antigen produced by a tumor cell of the subject.

Accordingly, in a further preferred embodiment, the method further comprises, following step i), administering an antigen produced by a tumor cell of the subject.

In a second aspect, the present invention provides a method of treating cancer in a mammalian subject which has been exposed to anti-cancer compounds, or has had at least part of the tumor removed by surgery, or has been exposed to radiotherapy, the method comprising administering to the subject an agent which inhibits the production of, limits the function of, and/or destroys, regulator cells, wherein the timing of administration of the agent is selected such that the activity of effector cells is not significantly reduced.

Restimulation of effector cells in a mammalian subject suffering from cancer can be achieved by increasing the quantity of tumor antigen within the subject. The addition of further tumor antigens enhances the effector cell response, thereby providing an opportunity to ablate or inhibit the regulators of these new effectors.

Accordingly, in a third aspect the present invention provides a method of treating cancer in a mammalian subject, the method comprising
i) administering a tumor antigen, which results in an increase in the number of, and/or activates, effector cells directed against the tumor antigen, and
ii) subsequently administering to the subject an agent which inhibits the production of, limits the function of, and/or destroys, regulator cells,
   wherein the timing of administration of the agent is selected such that the activity of the effector cells is not significantly reduced.

In a preferred embodiment of the third aspect, the agent is administered approximately when CD8+CD4-T cell numbers have peaked in response to the administration of the tumor antigen.

In a further preferred embodiment of the third aspect, the agent is administered approximately when the number of tumor cells has begun to stabilize or decrease following the administration of the tumor antigen.

In another preferred embodiment of the third aspect, the agent is administered approximately when the number of circulating tumor antigens has begun to stabilize or decrease following the administration of the tumor antigen.

Preferably, the antigen is provided to the subject by administering a vaccine comprising the tumor antigen and a pharmaceutically acceptable carrier. More preferably, the vaccine further comprises an adjuvant.

In another embodiment the antigen is provided to the subject by administering a DNA vaccine encoding the antigen.

In yet another embodiment, the antigen is provided to the subject by the consumption of a transgenic plant expressing the antigen.

Preferably, the agent used in regulator cell ablation is selected from the group consisting of anti-proliferative drugs, radiation, and antibodies which inhibit the down regulation activity of the regulator cells. Preferably, the anti-proliferative drug is selected from the group consisting of vinblastine and anhydro vinblastine. Notably, in some instances, particularly with regard to the first aspect, the anti-cancer compound and the agent can in fact be the same molecule.

Examples of preferred antibodies include, but are not limited to, anti-CD4+, anti-CTLA-4 (cytotoxic lymphocyte-associated antigen-4), anti-GITR (glucocorticoid-induced tumor necrosis factor receptor), anti-CD28 and anti-CD25.

As would be readily appreciated by those skilled in the art, the methods of the present invention may be repeated to provide a more complete treatment.

Preferably, the mammalian subject is a human.

In a further aspect, the present invention provides the use of an agent which inhibits the production of, limits the function of, and/or destroys, regulator cells for the manufacture of a medicament for administering to a mammalian subject with cancer, wherein the agent is administered subsequent to the subject's tumor load having been reduced which allows the number and/or activity of effector cells, directed against a tumor antigen, to increase in response to tumor antigen based stimulation of effector cells, and wherein the timing of administration of the agent is selected such that the activity of the effector cells is not significantly reduced.

In another aspect, the present invention provides the use of an anti-cancer compound for the manufacture of a medicament for administering to a mammalian subject with cancer, wherein the anti-cancer compound reduces tumor load which allows the number and/or activity of effector cells, directed against a tumor antigen, to increase in response to tumor antigen based stimulation of effector cells, and wherein the subject is subsequently administered with an agent which inhibits the production of, and/or destroys, regulator cells, and wherein the timing of administration of the agent is selected such that the activity of the effector cells is not significantly reduced.

In yet a further aspect, the present invention provides the use of an agent which inhibits the production of, limits the function of, and/or destroys, regulator cells for the manufacture of a medicament for administering to a mammalian subject with cancer, wherein the mammalian subject has been exposed to anti-cancer compounds, or has had at least part of the tumor removed by surgery, or has been exposed to radiotherapy, and wherein the timing of administration of the agent is selected such that the activity of the effector cells is not significantly reduced.

In yet another aspect, the present invention provides the use of an agent which inhibits the production of, limits the function of, and/or destroys, regulator cells for the manufacture of a medicament for administering to a mammalian subject with cancer, wherein the subject has previously been administered with a tumor antigen which results in an increase in the number of, and/or activates, effector cells directed against the tumor antigen, and wherein the timing of administration of the agent is selected such that the activity of the effector cells is not significantly reduced.

In another aspect, the present invention provides the use of a tumor antigen for the manufacture of a medicament for administering to a mammalian subject with cancer producing the tumor antigen, wherein the tumor antigen which results in an increase in the number of, and/or activates, effector cells directed against the tumor antigen, and wherein the subject is subsequently administered with an agent which inhibits the production of, limits the function of, and/or destroys, regulator cells, and wherein the timing of administration of the agent is selected such that the activity of the effector cells is not significantly reduced.

In each aspect outlined above, ablation of the “regulator” population allows the “effector” population to reduce or eradicate tumor cells as any down regulation of effectors has been removed.

As will be apparent, preferred features and characteristics of one aspect of the invention are applicable to many other aspects of the invention.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

The invention is hereinafter described by way of the following non-limiting examples and with reference to the accompanying figures.

### BRIEF DESCRIPTION OF THE ACCOMPANYING DRAWINGS

FIG. 1. Effect of Vb treatment on mesothelioma area. Day 0=Mesothelioma inoculation measurements with callipers at time points on day 14 and beyond. Tumor area means and standard error of the means are shown.

### DETAILED DESCRIPTION OF THE INVENTION

Definitions

As used herein the term “treating” or “treat” means a reduction in tumor load is achieved. In some aspects of the present invention, one step involves reducing tumor load through the use of known techniques, such as surgery, radiotherapy or chemotherapy, and then administering the agent at the appropriate time. In these aspects, the term “treating” or “treat” is used to indicate that the tumor load in the subject is further reduced by the method of the present invention. Most preferably, the tumor load is completely eradicated.

As used herein, the term “tumor load” generally refers to the number of cancerous cells in a subject at any given time.

“Regulator cells” include, but are not necessarily limited to, a subpopulation of CD4+ T cells. Such cells may also be referred to in the art as “suppressor cells”. Regulator cells may either act directly on effector cells or may assert their affects upon effector cells through other mechanisms.

CD4+ cells express the marker known in the art as CD4. Typically, the term “CD4+ T cells” as used herein does not refer to cells which also express CD8. However, this term can include T cells which also express other antigenic markers such as CD25.

“Effector cells” include, but are not necessarily limited to, the T cell population known as CD8+ cells.

As used herein, the term “ablate” or “ablation” when referring to the exposure of the “regulator cells” to the agent means that the number, and/or activity, of regulator cells is down-regulated by the agent. Most preferably, the number, and/or activity, of regulator cells is completely eradicated by the agent.

As is known in the art, a cancer is generally considered as uncontrolled cell growth. The methods of the present invention can be used to treat any cancer including, but not limited to, carcinoma, lymphoma, blastoma, sarcoma, and leukemia. More particular examples of such cancers include breast cancer, prostate cancer, colon cancer, squamous cell cancer, small-cell lung cancer, non-small cell lung cancer, ovarian cancer, cervical cancer, gastrointestinal cancer, pancreatic cancer, glioblastoma, liver cancer, bladder cancer, hepatoma, colorectal cancer, uterine cervical cancer, endometrial carcinoma, salivary gland carcinoma, mesothelioma, kidney cancer, vulval cancer, thyroid cancer, hepatic carcinoma, skin cancer, melanoma, brain cancer, ovarian cancer, neuroblastoma, myeloma, various types of head and neck cancer, acute lymphoblastic leukemia, acute myeloid leukemia, Ewing sarcoma and peripheral neuroepithelioma.

Unless otherwise indicated, the recombinant DNA and immunological techniques utilized in the present invention are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present), and are incorporated herein by reference.

Agents Which Inhibit the Production of, Limit the Function of, And/Or Destroy, Regulator Cells

The agent can be any factor or treatment which selectively or non-selectively results in the destruction, or the inhibition of the production, of regulator cells. For example, a CD4+ specific antibody could be used to specifically target CD4+ T cells. However, in some instances a non-selective agent could be used, such as an anti-proliferative drug or radiation, both of which destroy dividing cells.

The term “anti-proliferative drug” is a term well understood in the art and refers to any compound that destroys dividing cells or inhibits them from undergoing further proliferation. Anti-proliferative drugs include, but are not limited to, mechlorethamine, cyclophosphamide, ifosfamide, melphalan, chlorambucil, hexamethyl-melamine, thiotepa, busulfan, carmustine, lomustine, semustine, streptozocin, dacarbazine, methotrexate, fluorouracil, floxuridine, cytarabine, mercaptopurine, thioguanine, pentostatin, vinblastine, anhydro vinblastine, vincristine, etoposide, teniposide, dactinomycin, daunorubicin, doxorubicin, bleomycin, plicamycin, mitomycin, L-asparaginase, cisplatin, mitoxantrone, hydroxyurea, procarbazine, mitotane, aminoglutethimide, prednisone, hydroxyprogesterone caproate, medroprogesterone acetate, megestrol acetate, diethylstilbestrol, ethinyl estradiol, tamoxifen, testosterone propionate, radioactive isotopes, ricin A chain, taxol, diphtheria toxin and pseudomonas exotoxin A.

The agent can be administered as standard dosages as used in the art. In one embodiment, the agent is administered as a single bolus injection. In another embodiment, the agent is administered by infusion over a period of, for example, 24 hours.

Recent studies have suggested that CD4+CD25+ T cells play an important role in regulating immune cells directed against self antigens (Salomon et al., 2000; Suri-Payer and Cantor, 2001). Furthermore, targeted ablation of CD4+CD25+ T cells has been shown to enhance the ability of an animal to control tumor growth (Onizuka et al., 1999; Shimizu et al., 1999; Sutmuller et al., 2001). Accordingly, CD4+CD25+ T cells could be acting as regulator cells as used herein. The activity of CD4+CD25+ T cells can be downregulated by anti-GITR, anti-CD28 and/or anti-CTLA-4 (Read et al., 2000; Takahashi et al., 2000; Shimizu et al., 2002). Thus, these antibodies may be useful as agents for use in the methods of the present invention.

Timing of Exposing the Subject To the Agent

As outlined above, the present invention relies on the observation that the relative number of effector cells expands in response to an antigen before the regulator cells. Accordingly, as used herein, the term “the activity of the effector cells is not significantly reduced” means that the timing of the administration of the agent is such that the agent exerts a proportionally greater effect against the regulator cells than the effector cells. It is clearly preferred that the agent is administered at a time when the ratio of effect against the regulator cells to the effect against effector cells is greatest.

Some aspects of the present invention rely on reducing the tumor load before selecting for the effector cells. A number of avenues exist which enable the effector cells to be selected through ablation of the regulator cells. One comprises administering the agent approximately when CD8+CD4-T cell numbers have peaked in response to tumor antigen based stimulation of effector cells following the reduction in tumor load through techniques such as, but not limited to radiotherapy, chemotherapy and surgery. Another avenue comprises administering the agent approximately when the tumor cell numbers are decreasing and when regulator cell numbers are increasing in response to tumor antigen based stimulation of effector cells following the reduction in tumor load through techniques such as, but not limited to radiotherapy, chemotherapy and surgery.

In most instances, the time point that the agent is to be administered will need to be empirically determined in subjects at different stages of cancer as their immune response kinetics may vary. Other factors such as the general health of the subject and/or the genetic makeup of the subject will also impact upon when is the appropriate time to administer the agent.

Techniques known in the art can be used to monitor the growing population of effector cells following a reduction in the tumor load, or the stimulation of an immune response by exposing the subject to further tumor antigens. For instance, the production of acute phase proteins, such as c-reactive protein (for example as disclosed in Price et al. 1987), will indicate the presence of an immune response to the tumor antigen.

Techniques known in the art can also be used to monitor the growing population of regulator cells following a reduction in the tumor load, or the stimulation of an immune response by exposing the subject to further tumor antigens. Some of these techniques are discussed below.

Serial blood samples can be collected and quantitatively screened for all CD4+ subsets by FACS analysis. This FACS monitoring will need to be maintained until the regulator cells begin clonally expanding in response to tumor antigens, whether produced by the tumor or administered to the subject. Other possible assays for monitoring the growing population of regulator cells include lymphocyte proliferation/activation assays and various cytokine level assays (for example an assay for IL-4, IL-6 or IL-10).

Another avenue of determining the time point for administering the agent is to monitor the tumor load. It is envisaged that the tumor load decreases due to the activity of the effector cells, however, the subsequent increase in regulator cells would down-regulate the effector cells resulting in a slowing of the tumor load decrease. Accordingly, the agent could be administered approximately prior to the slowing of the decrease in tumor load. Techniques known in the art, for example RT-PCR or antibody detection, of markers expressed by the tumor, could be used to measure tumor load in these circumstances. Examples of suitable tumor antigen marker assays include, but are not limited to, for AFP (marker for hepatocellular carcinoma and germ-cell tumors), CA 15-3 (marker for numerous cancers including breast cancer), CA 19-9 (marker for numerous cancers including pancreatic cancer and biliary tract tumors), CA 125 (marker for various cancers including ovarian cancer), calcitonin (marker for various tumors including thyroid medullary carcinoma), catecholamines and metabolites (phaeochromoctoma), CEA (marker for various cancers including colorectal cancers and other gastrointestinal cancers), hCG/beta hCG (marker for various cancers including germ-cell tumors and choriocarcinomas), 5HIAA in urine (carcinoid syndrome), PSA (prostate cancer), sertonin (carcinoid syndrome) and thyroglobulin (thyroid carcinoma).

Monitoring may need to be very frequent, for example as often as every few hours, to ensure the correct time point is selected for administration of the agent. Preferably, the monitoring is conducted at least every 48 hours. More preferably, the monitoring is conducted at least every 24 hours.

Optimally, the monitoring is continued to determine the affect of the agent. Insufficient ablation, re-emergence of the regulator cells or increases in tumor load will mean that the method of the present invention should be repeated. Such repeated cycles of treatment may generate immunological memory. It is therefore possible that the present invention, used in repetitive mode, may provide some prophylactic protective effect.

C-Reactive Protein

C-reactive protein (CRP) is an important acute phase response protein, and its concentration in serum may increase as much as 1,000-fold during the acute phase response. CRP is a pentamer consisting of five identical subunits, each having a molecular weight of about 23,500.

CRP levels have previously been shown to be markers of the recurrence of a cancer (Hosotsubo et al. 2000; Mahmoud and Rivera, 2002). C-reactive protein levels can be determined using techniques known in the art, these include, but are not limited to, those disclosed in Senju et al. (1983), Price et al. (1987) and Eda et al. (1998).

Tumor Antigens

As used herein, an “antigen” is any polypeptide sequence that contains an epitope which is capable of producing an immune response against the cancer. Preferably, the antigen will comprise a sequence which is highly selective for the cancer cell. It is envisaged that cells of a particular cancer could be characterized and an antigen produced which matches the sequences of the cancer to maximise the possibility of an effective immune response.

Antigens which are capable of raising an immune response against a cancer cell are well known in the art. Certain tumor antigens can be recognized and targeted by the immune system. This property may be due to overexpression by the tumor tissue. Some of these antigens can be detected in normal tissue. The tumor antigens targeted by T cells are generally proteins that are processed intracellularly and presented as short peptide fragments bound in the groove of the tumor MHC class I molecule to be recognized by CD8⁺ cytotoxic T lymphocytes. The mere presence of a tumor antigen is not always sufficient to trigger an immune response. Co-stimulatory molecules such as B7.1 are sometimes required. Once antigen-specific T cells are stimulated, they are capable of recognizing and destroying the tumor. The conditions needed for the activation of antigen-specific T cells are stringent, but are open to genetic manipulation of target tumor cells and T cells.

The antigen can be provided in any manner known in the art which leads to an immune response. An antigen can be, for example, native, recombinant or synthetic. Native antigens can be prepared, for example, by providing cell lysates of a tumor cell.

The antigen can be provided as isolated polypeptides in a vaccine composition. In this instance the antigen can be purified from tumor cells, expressed and isolated from recombinant cells, or synthetically produced using a peptide synthesizer.

Vaccines

Vaccines may be prepared from one or more antigens. The preparation of vaccines which contain an antigen is known to one skilled in the art. Typically, such vaccines are prepared as injectables, or orals, either as liquid solutions or suspensions; solid forms suitable for solution in, or suspension in, liquid prior to injection or oral consumption may also be prepared. The preparation may also be emulsified, or the protein encapsulated in liposomes. The antigen is often mixed with carriers/excipients which are pharmaceutically acceptable and compatible with the active ingredient. Suitable carriers/excipients are, for example, water, saline, dextrose, glycerol, ethanol, or the like and combinations thereof.

In addition, if desired, the vaccine may contain minor amounts of auxiliary substances such as wetting or emulsifying agents, pH buffering agents, and/or adjuvants which enhance the effectiveness of the vaccine.

As used herein, the term “adjuvant” means a substance that non-specifically enhances the immune response to an antigen. Examples of adjuvants which may be effective include but are not limited to: N-acetyl-muramyl-L-threonyl-D-isoglutamine (thr-MDP), N-acetyl-nor-muramyl-L-alanyl-D-isoglutamine (CGP 11637, referred to as nor-MDP), N-acetylmurarnyl-L-alanyl-D-isoglutaminyl-L-alanine-2-(1′-2′-dipalmitoyl-sn-glycero-3-hydroxyphosphoryloxy)-ethylamine (CGP 19835A, referred to as MTP-PE), and RIBI, which contains three components extracted from bacteria, monophosphoryl lipid A, trehalose dimycolate and cell wall skeleton (MPL+TDM+CWS) in a 2% squalene/Tween 80 emulsion. Further examples of adjuvants include aluminum hydroxide, aluminum phosphate, aluminum potassium sulfate (alum), bacterial endotoxin, lipid X, Corynebacterium parvum (Propionobacterium acnes), Bordetella pertussis, polyribonucleotides, sodium alginate, lanolin, lysolecithin, vitamin A, saponin, liposomes, levamisole, DEAE-dextran, blocked copolymers or other synthetic adjuvants. Such adjuvants are available commercially from various sources, for example, Merck Adjuvant 65 (Merck and Company, Inc., Rahway, N.J.) or Freund's Incomplete Adjuvant and Complete Adjuvant (Difco Laboratories, Detroit, Mich.).

The proportion of antigen and adjuvant can be varied over a broad range so long as both are present in effective amounts. For example, aluminum hydroxide can be present in an amount of about 0.5% of the vaccine mixture (Al₂O₃ basis). Conveniently, the vaccines are formulated to contain a final concentration of antigenic polypeptide in the range of from 0.2 to 200 μg/ml, preferably 5 to 50 μg/ml, most preferably 15 μg/ml.

After formulation, the vaccine may be incorporated into a sterile container which is then sealed and stored at a low temperature, for example 4° C., or it may be freeze-dried. Lyophilisation permits long-term storage in a stabilised form.

The vaccines are conventionally administered parenterally, by injection, for example, either subcutaneously or intramuscularly. Additional formulations which are suitable for other modes of administration include suppositories and, in some cases, oral formulations. For suppositories, traditional binders and carriers may include, for example, polyalkylene glycols or triglycerides; such suppositories may be formed from mixtures containing the active ingredient in the range of 0.5% to 10%, preferably 1% to 2%. Oral formulations include such normally employed excipients as, for example, pharmaceutical grades of mannitol, lactose, starch, magnesium stearate, sodium saccharine, cellulose, magnesium carbonate, and the like. These compositions take the form of solutions, suspensions, tablets, pills, capsules, sustained release formulations or powders and contain 10% to 95% of active ingredient, preferably 25% to 70%. Where the vaccine composition is lyophilised, the lyophilised material may be reconstituted prior to administration, e.g. as a suspension. Reconstitution is preferably effected in buffer.

Capsules, tablets and pills for oral administration to a patient may be provided with an enteric coating comprising, for example, Eudragit “S”, Eudragit “L”, cellulose acetate, cellulose acetate phthalate or hydroxypropylmethyl cellulose.

DNA Vaccination

DNA vaccination involves the direct in vivo introduction of DNA encoding an antigen into tissues of a subject for expression of the antigen by the cells of the subject's tissue. Such vaccines are termed herein “DNA vaccines” or “nucleic acid-based vaccines”. DNA vaccines are described in U.S. Pat. No. 5,939,400, U.S. Pat. No. 6,110,898, WO 95/20660 and WO 93/19183, the disclosures of which are hereby incorporated by reference in their entireties. The ability of directly injected DNA that encodes an antigen to elicit a protective immune response has been demonstrated in numerous experimental systems (see, for example, Conry et al., 1994; Cardoso et al., 1996; Cox et al., 1993; Davis et al.,1993; Sedegah et al., 1994; Montgomery et al., 1993; Ulmer et al., 1993; Wang et al., 1993; Xiang et al., 1994; Yang et al., 1997).

To date, most DNA vaccines in mammalian systems have relied upon viral promoters derived from cytomegalovirus (CMV). These have had good efficiency in both muscle and skin inoculation in a number of mammalian species. A factor known to affect the immune response elicited by DNA immunization is the method of DNA delivery, for example, parenteral routes can yield low rates of gene transfer and produce considerable variability of gene expression (Montgomery et al., 1993). High-velocity inoculation of plasmids, using a gene-gun, enhanced the immune responses of mice (Fynan et al., 1993; Eisenbraun et al., 1993), presumably because of a greater efficiency of DNA transfection and more effective antigen presentation by dendritic cells. Vectors containing the nucleic acid-based vaccine of the invention may also be introduced into the desired host by other methods known in the art, e.g., transfection, electroporation, microinjection, transduction, cell fusion, DEAE dextran, calcium phosphate precipitation, lipofection (lysosome fusion), or a DNA vector transporter.

Vaccines Derived from Transgenic Plants

Transgenic plants producing a antigenic polypeptide can be constructed using procedures well known in the art. A number of plant-derived edible vaccines are currently being developed for both animal and human pathogens (Hood and Jilka, 1999). Immune responses have also resulted from oral immunization with transgenic plants producing virus-like particles (VLPs), or chimeric plant viruses displaying antigenic epitopes (Mason et al., 1996; Modelska et al., 1998; Kapustra et al., 1999; Brennan et al., 1999). It has been suggested that the particulate form of these VLPs or chimeric viruses may result in greater stability of the antigen in the stomach, effectively increasing the amount of antigen available for uptake in the gut (Mason et al. 1996, Modelska et al. 1998).

### EXAMPLES

### Example 1

Malignant mesothelioma in humans is an asbestos induced, incurable tumor that has a particularly poor prognosis. Average time from diagnosis to death being only eight months (Musk and Woodward, 1982). A murine model was developed by researchers in the Department of Medicine at the University of Western Australia to further the development of suitable interventions (Davies et al., 1992). The murine tumors exhibit morphological features, distributions, and mode of spread in body cavities consistent with the human clinical situation.

Day 0 was defined as the day of tumor cell inoculation with 1×10⁶ AE 17 cells subcutaneously. The AE 17 tumor cell line was derived from C57/B16 mice and inoculated back into the same strain. This point will be referred to as a tumor challenge henceforth. Three groups of 5 mice were all challenged in this way. Subsequently the first of the groups was given a single ip dose of vinblastine (Vb) at 6 mg/kg body weight exactly 14 days post tumor challenge (Vb 19/11/01 group). The second group of 5 mice were similarly treated the next day; 15 days post tumor challenge (Vb 20/11/01). The third group received no Vb treatment (No treatment/control group).

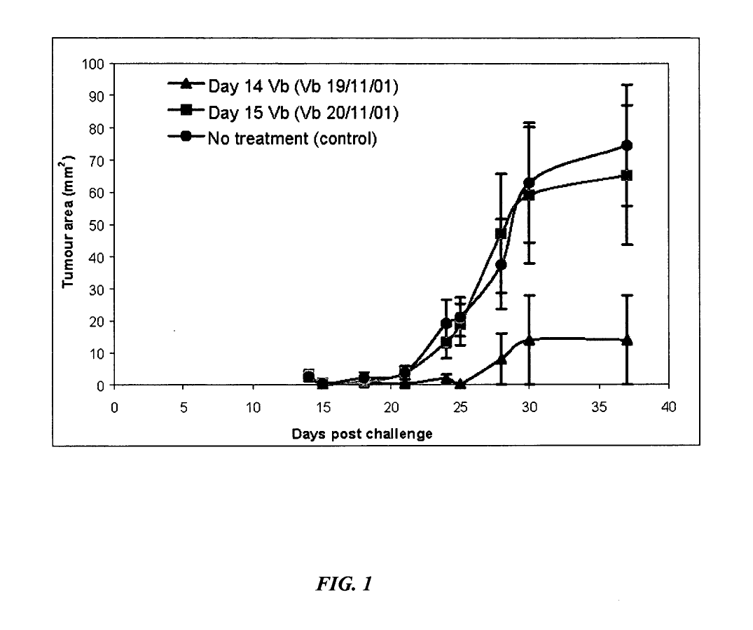

Tumor measurements were taken from Day 14 onwards with callipers (2 diameters at right angles to each other in mm's). These two diameters are then multiplied together to give a mm² value that is known from past experience to correlate well with both tumor volume and tumor mass. The resultant data from this experiment is presented in Table 1 and FIG. 1.

Clearly the mice receiving the day 14 post challenge single Vb treatment are heavily protected from tumor development. In contrast the control and day 15 Vb treatment groups show significant tumor development in 4/5 mice. The data from this shows that a narrow “therapeutic window” for Vb treatment was shown and an 80% effectiveness at this time point.

### Example 2

A prostate cancer patient with elevated levels of PSA associated with the cancer is subjected to a standard chemotherapy regime involving cyclophosphamide (for example, as described by Casciato and Lowitz, 1995). Upon completion of the chemotherapy the patient is monitored for the PSA marker. Such monitoring should preferably occur at least every 24 hours after the completion of chemotherapy and continue for at least one month.

If PSA marker levels begin to elevate it will be clear that the chemotherapy is not completely successful. PSA levels are continued to be monitored until levels of PSA are decreasing. This indicates that the effector cells are beginning to control the new cancerous cells. At approximately this point vinblastine is administered to the patient at a standard dose such as 3-4 mg/m² intravenously (Casciato and Lowitz, 1995). Vinblastine will target dividing cells, such as the regulator cells, which begin to clonally expand to control effector cell levels.

The patient is further monitored for PSA levels to ensure that the cancer is controlled. Treatment is repeated if necessary.

### Example 3

A breast cancer patient with elevated levels of CA 15-3 (epitope of Polymorphic Epithelial Mucin) associated with the cancer is subjected to surgery in an attempt to remove the tumor. Upon completion of the chemotherapy the patient is monitored for the CA 15-3 marker. Such monitoring should preferably occur at least every 24 hours after surgery and continue for at least one month.

If CA 15-3 marker levels begin to elevate it is clear that all of the tumor cells are not removed by the surgery. CA 15-3 levels are continued to be monitored until levels of CA 15-3 are decreasing. This indicates that the effector cells are beginning to control the new cancerous cells. The patient is then subjected to radiation therapy to target dividing cells, for example provided as megavoltage gamma irradiation to the entire breast (about 4500-5000 cGy) (Casciato and Lowitz, 1995).

The patient is further monitored for CA 15-3 levels to ensure that the cancer is controlled. Treatment is repeated if necessary.

### Example 4

An acute lymphoblastic leukemia patient is subjected to a standard chemotherapy regime involving vincristine and prednisone (Casciato and Lowitz, 1995). Upon completion of the chemotherapy the patient is monitored for c-reactive protein levels as generally described by Price et al. (1987). Such monitoring should preferably occur at least every 24 hours after the completion of chemotherapy and continue for at least one month.

If c-reactive protein levels begin to elevate it will indicate that the patients immune system is mounting an immune response to an antigen, and that all cancerous cells are not eliminated by the chemotherapy. Naturally, the patient should be examined for any indications of, for example, viral or bacterial infections which may contribute to the elevated c-reactive protein levels. In the absence of such indications, c-reactive protein levels are continued to be monitored until levels of c-reactive protein peak and begin to decrease. This indicates that the effector cells are beginning to control the new cancerous cells. At this point anti-CD4+ antibodies which at least target part of the regulator cell population are administered to the patient at a standard doses such as 300 mg.

The patient is further monitored for c-reactive protein levels to ensure that the cancer is controlled. Treatment repeated if necessary.

### Example 5

A melanoma patient is examined for MAGE-3 which is expressed in approximately 76% of melanoma patients. If MAGE-3 expression is detected the patient is injected with a suitable MAGE-3 peptide in a vaccine composition as generally described by Coulie et al. (2001).

The patient is then monitored for CD8+CD4-T cell numbers, preferably at least every 24 hours for at least one month. Approximately when CD8+CD4-T cell numbers have peaked in response to tumor antigen based stimulation of effector cells the patient is administered with vinblastine at a standard dose such as 3-4 mg/m² intravenously. Vinblastine will target dividing cells, such as regulator cells which clonally expand to control effector cell levels.

The patient is further monitored for cancerous cells, such as using the S100 protein as a marker (Casciato and Lowitz, 1995). If further melanoma cells are detected the treatment can be repeated.

As the skilled addressee would be aware, the general methods used to reduce tumor load, target regulator cells, and determine the timing of administering the agent are typically interchangeable for any given treatment. Naturally, standard techniques of, for example chemotherapy, radiation therapy, surgery and tumor cell marker detection, will preferably be performed as already used in the art (for example see (Casciato and Lowitz, 1995).

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

All publications discussed above are incorporated herein in their entirety.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority of each claim of this application.

### REFERENCES

Brennan, F. R., Bellaby, T., Helliwell, S. M., Jones, T. D., Kamstrup, S., Dalsgaard, K., Flock, J-I. and Hamilton, W. D. O. (1999) J. Virol. 73, 930-938.

Cardoso, A. I., Blixenkrone-Moller, M., Fayolle, J., Liu, M., Buckland, R. and Wild, T. F. (1996) Virology 225, 293-299.

Casciato, D. A. and Lowitz, B. B. (1995) Manual of Clinical Oncology. Third Edition. Little Brown and Company, Boston.

Conry, R. M., LoBuglio, A. F., Kantor, J., Schlom, J., Loechel, F., Moore, S. E., Sumerel, L. A., Barlow, D. L., Abrams, S. and Curiel, D. T. (1994) Cancer Res. 54, 1164-1168.

Coulie, P. G., Karanikas, V., Colau, D., Lurquin, C., Landry, C., Marchand, M., Dorval, T., Brichard, V. and Boon, T. (2001) Proc. Natl. Acad. Sci., USA 98, 10290-10295.

Cox, G. J., Zamb, T. J. and Babiuk, L. A. (1993) J. Virol. 67, 5664-5667.

Davis, H. L., Michel, M. L. and Whalen, R. G. (1993) Human Mol. Genet. 2, 1847-1851.

Davies, M. R., Manning, L. S., Whitaker, D., Garlepp, M. J. and Robinson, B. W. (1992). Int. J. Cancer 52, 881-886.

Eda, S., Kaufmann, J., Roos, W. and Phol, S. (1998). J. Clin. Lab. Anal. 12, 137-144.

Eisenbraun, M. D., Fuller, D. H. and Haynes, J. R. (1993) DNA Cell Biol. 12, 791-797.

Fynan, E. F., Webster, R. G., Fuller, D. H., Haynes, J. R., Santoro, J. C. and Robinson, H. L. (1993) Proc. Natl. Acad. Sci. U.S.A. 90, 11478-11482.

Hood, E. E. and Jilka, J. M. (1999) Current Opin. Biotech. 10, 382-386.

Hosotsubo, K. K., Nishimura, M. and Nishimura, S. (2000) Crit. Care 4, 180-187.

Kapustra, J., Modelska, A., Figlerowicz, M., Pniewski, T., Letellier, M., Lisowa, O., Yusibov, V., Koprowski, H., Plucienniczak, A. and Legocki, A. B. (1999) FASEB J. 13, 1796-1799.

Kruse, C. A., Lillehei, K. O., Mitchell, D. H., Kleinschmidt-DeMasters, B. and Bellgrau, D. (1990) Proc. Natl. Acad. Sci. U.S.A. 87, 9577-9581.

Mahmoud, F. A. and Rivera, N. I. (2002) Curr. Oncol. Rep. 4, 250-255.

Mason H. S., Ball J. M., Shi J. J., Jiang X., Estes M. K. and Amtzen C. J. (1996) Proc. Natl. Acad. Sci. USA 93, 5335-5340.

Modelska, A., Dietzschold, B., Sleysh, N., Fu, Z. F., Steplewski, K., Hooper, D. C., Koprowski, H. and Yusibov, V. (1998) Proc. Natl. Acad. Sci. U.S.A. 95, 2481-2485.

Montgomery, D. L., Shiver, J. W., Leander, K. R., Perry, H. C., Friedman, A., Martinez, D., Ulmer, J. B., Donnelly, J. J. and Lui, M. A. (1993) DNA Cell Biol. 12, 777-783.

Musk, A. W. and Woodward, S. D. (1982) Aust. NZ J. Med. 12, 229-232.

Onizuka, S., Tawara, I., Shimizu, J., Sakaguchi, S., Fujita, T. and Nakayama, E. (1999) Cancer Res. 59, 3128-3133.

Price, C. P., Trull, A. K., Berry, D. and Gorman, E. G. (1987) J. Immunol. Methods 99, 205-211,

Read, S., Malmstrom, V. and Powrie, F. (2000) J. Exp. Med. 192, 295-302.

Sakaguchi, S., Sakaguchi, N., Shimizu, J., Yamazaki, S., Sakihama, T., Itoh, M., Kuniyasu, Y., Nomura, T., Toda, M. and Takahashi, T. (2001) Immunological Reviews 182, 18-32.

Salomon, B., Lenschow, D. J., Rhee, L., Ashourian, N., Singh, B., Sharpe, A. and Bluestone, J. A. (2000) Immunity 12, 431-440.

Sedegah, M., Hedstrom, R., Hobart, P. and Hoffman, S. L. (1994) Proc. Natl Acad. Sci. U.S.A. 91, 9866-9870.

Senju, O., Takagi, Y., Gomi, K., Ishii, N., Mochizuki, S. Ishii, N. et al. (1983) Jap. J. Clin. Lab. Automat. 8, 161-165.

Shimizu, J., Yamazaki, S. and Sakaguchi, S. (1999) J. Immunol. 163, 5211-5218.

Shimiszu, J., Yamazaki, S., Takahashi, T., Ishida, Y. and Sakaguchi, S. (2002) Nature Immunol. 3, 135-142.

Suri-Payer, E. and Cantor, H. (2001) J. Autoimmunity 16, 115-23.

Sutmuller, R. P., van Duivenvoorde, L. M., van Elsas, A., Schumacher, T. N., Wildenberg, M. E., Allison, J. P., Toes, R. E., Offringa, R. and Melief, C. J. (2001) J. Exp. Med. 194, 823-832.

Takahashi, T., Tagami, T., Yamazaki, S., Uede, T., Shimizu, J., Sakaguchi, N., Mak, T. W. and Sakaguchi, S. (2000) J. Exp. Med. 192, 303-310.

Ulmer, J. B., Donnelly, J. J., Parker, S. E., Rhodes, G. H., Felgner, P. L., Dwarki, V. J., Gromkowski, S. H., Deck, R. R., DeWitt, C. M., Friedman, A. et al. (1993) Science 259, 1745-1749.

Wang, B., Ugen, K. E., Srikantan, V., Agadjanyan, M. G., Dang, K., Refaeli, Y., Sato, A. I., Boyer, J., Williams, W. V. and Weiner, D. B. (1993) Proc. Natl. Acad. Sci. U.S.A. 90, 4156-4160.

Xiang, Z. Q., Spitalnik, S., Tran, M., Wunner, W. H., Cheng, J. and Ertl, H. C. (1994) Virology 199, 132-140.

Yang, K., Mustafa, F., Valsamakis, A., Santoro, J. C., Griffin, D. E. and Robinson, H. L. (1997) Vaccine 15, 888-891.

TABLE 1

| | | 19-Nov | 20-Nov | 23-Nov | 26-Nov | 29-Nov | 30-Nov | 3-Dec | 5-Dec | 7-Dec |
|---|---|---|---|---|---|---|---|---|---|---|
| Treatment | Animal # | 14* | 15* | 18* | 21* | 24* | 25* | 28* | 30* | 37* |
| Vb Nov. 19, 2001 | 1 | 4 | 0.5 | 0 | 0.5 | 0 | 0 | 0 | 0 | 0 |
| Day 14 | 2 | 4 | 0.5 | 2 | 0.5 | 6 | 1 | 0.5 | 0 | 0 |
| | 3 | 4 | 0.5 | 1 | 1 | 4 | 0.5 | 40 | 70 | 70 |
| | 4 | 4 | 0 | 0 | 0 | 0 | 0 | 0 | 0 | 0 |
| | 5 | 0.5 | 0 | 0.5 | 0 | 0 | 0 | 0 | 0 | 0 |
| | | 3.3 | 0.3 | 0.7 | 0.4 | 2 | 0.3 | 8.1 | 14 | 14 |
| Vb Nov. 20, 2001 | 1 | 0.5 | 1 | 4 | 9 | 16 | 20 | 42 | 56 | 81 |
| Day 15 | 2 | 0.5 | 0.5 | 0.5 | 0.5 | | | | | |
| | 3 | 0.5 | 0 | 0 | 0.5 | 0.5 | 0.5 | 0.5 | 0.5 | 0.5 |
| | 4 | 4 | 0.5 | 0.5 | 8 | 25 | 30 | 56 | 90 | 90 |
| | 5 | 9 | 0.5 | 0 | 0.5 | 12 | 25 | 90 | 90 | 90 |
| | | 2.9 | 0.5 | 1 | 3.7 | 13.4 | 18.9 | 47.1 | 59.1 | 65.4 |
| No treatment | 1 | 0.5 | 0.5 | 0.5 | 1 | 30 | 20 | 42 | 99 | 99 |
| Control | 2 | 0.5 | 0 | 0 | 2 | 6 | 20 | 20 | 42 | 100 |
| | 3 | 4 | 0 | 0.5 | 0 | 0 | 0 | 0 | 0 | 0 |
| | 4 | 4 | 0.5 | 1 | 4 | 20 | 30 | 42 | 90 | 90 |
| | 5 | 4 | 1 | 9 | 12 | 40 | 36 | 84 | 84 | 84 |
| | | 2.6 | 0.4 | 2.2 | 3.8 | 19.2 | 21.2 | 37.6 | 63 | 74.6 |

1. A method of treating cancer in a mammalian subject comprising
i) reducing tumour load in the subject,
ii) allowing the number and/or activity of effector cells, directed against a tumour antigen, to increase in response to tumour antigen based stimulation of effector cells, and
iii) administering to the subject an agent which inhibits the production of, limits the function of, and/or destroys, regulator cells,
wherein the timing of administration of the agent is selected such that the activity of the effector cells is not significantly reduced.

2. The method of claim 1, wherein fluctuations in the levels of c-reactive protein in the subject are monitored to determine when the agent is administered.

3. The method of claim 2, wherein the agent is administered when the levels of c-reactive protein begin to decrease.

4. The method of claim 1, wherein the agent is administered approximately when CD8+CD4-T cell numbers have peaked in response to tumour antigen based stimulation of effector cells.

5. The method of claim 1, wherein the agent is administered when the tumour cell numbers are decreasing and regulator cell numbers are increasing.

6. The method of claim 1, wherein tumour load is reduced by removing at least some of the cancer cells by surgery.

7. The method of claim 1, wherein the tumour load is reduced by administering an anti-cancer compound.

8. The method of claim 7, wherein the anti-cancer compound is vinblastine or anhydro vinblastine.

9. The method of claim 1, wherein the tumour load is reduced by exposing the subject to radiotherapy.

10. The method of claim 1, further comprising administering an antigen produced by a tumour cell of the subject.

11. A method of treating cancer in a mammalian subject with a reduced tumour load comprising administering to the subject an agent which inhibits the production of, limits the function of, and/or destroys, regulator cells, wherein the timing of administration of the agent is selected such that the activity of effector cells is not significantly reduced.

12. A method of treating cancer in a mammalian subject, the method comprising
i) administering a tumour antigen, which results in an increase in the number of, and/or activates, effector cells directed against the tumour antigen, and
ii) administering to the subject an agent which inhibits the production of, limits the function of, and/or destroys, regulator cells,
wherein the timing of administration of the agent is selected such that the activity of the effector cells is not significantly reduced.

13. The method of claim 12, wherein fluctuations in the levels of c-reactive protein in the subject are monitored to determine when the agent is administered.

14. The method of claim 13, wherein the agent is administered when the levels of c-reactive protein begin to decrease.

15. The method of claim 12, wherein the agent is administered when CD8+CD4-T cell numbers have peaked in response to the administration of the tumour antigen.

16. The method of claim 12, wherein the agent is administered approximately when the number of tumour cells has begun to stabilize or decrease following the administration of the tumour antigen.

17. The method of claim 12, wherein the agent is administered when the number of circulating tumour antigens has begun to stabilize or decrease following the administration of the tumour antigen.

18. The method of claim 12, wherein the antigen is provided to the subject by administering a vaccine composition comprising the tumour antigen and a pharmaceutically acceptable carrier.

19. The method of claim 18, wherein the vaccine composition further comprises an adjuvant.

20. The method of claim 12, wherein the antigen is provided to the subject by administering a DNA vaccine encoding the antigen.

21. The method of claim 12, wherein the antigen is provided to the subject by the consumption of a transgenic plant expressing the antigen.

22. The method of claim 1, wherein the agent is selected from the group consisting of anti-proliferative drugs, radiation, and an antibody which inhibits the regulator cells.

23. The method of claim 22, wherein the antibody is anti-CD4+.

24. The method according to any one of claims 1 to 23, wherein the method is repeated at least once.

25. The method according to claim 1, wherein the mammalian subject is a human.

26. A method for determining when an agent which inhibits the production of, limits the function of, and/or destroys, regulator cells, should be administered to a mammalian subject suffering from cancer comprising:
i) monitoring samples obtained from a subject, for any one of a) effector cell numbers or activity, b) regulator cell numbers or activity, or c) a marker of a) or b), wherein the samples are obtained after the number or activity of effector cells against the tumor in the subject have been increased; and
ii) analysing samples obtained every 48 hours or less after the increase in the number or activity of effector cells.

27. The method of claim 26, wherein the samples are obtained every 24 hours or less.

28. The method of claim 11, wherein tumor load is reduced by treatment with anti-cancer compounds, surgical removal of at least part of the tumor or expposure to radiotherapy.

29. (canceled)

30. (canceled)
